(19) United States
(12) Patent Application Publication
(10) Pub. No.: US 2005/0282281 A1
(43) Pub. Date: Dec. 22, 2005

(54) Recombinant baculoviral vector containing protein transduction domain gene

(76) Inventors: Soon-Young Paik, Seoul (KR); Jong-Sob Yoon, Seoul (KR); Seong-Karp Hong, Seongnam-si (KR)

Correspondence Address: ANDERSON, KILL & OLICK, P.C., 1251 AVENUE OF THE AMERICAS, NEW YORK,, NY, 10020-1182, US

(21) Appl. No.: 11/128,539

(22) Filed: May 13, 2005

(30) Foreign Application Priority Data
May 14, 2004 (KR) 10-2004-0034262

(51) Int. Cl. C12N 15/86

(52) U.S. Cl. 435/456

(57) ABSTRACT

A recombinant baculoviral vector, which comprises a vesicular stomatitis virus glycoprotein (VSVG) gene and a protein transduction domain (PTD) gene derived from HIV-1 (human immunodeficiency virus-1) Tat gene, is useful in a gene therapy owing to its excellent efficiencies in the delivery of a target gene to the host cell and expression of the target gene in the host cell.

### FIELD OF THE INVENTION

The present invention relates to a recombinant baculoviral vector having an excellent delivery efficiency of a target protein; and, more particularly, it pertains to a recombinant baculoviral vector comprising a vesicular stomatitis virus glycoprotein (VSVG) gene and a protein transduction domain (PTD) gene which is a part of Tat gene of HIV-1 (human immunodeficiency virus-1).

### BACKGROUND OF THE INVENTION

Recently, viral vectors such as adenovirus, adeno-associate virus, retrovirus, herpes simplex virus, lenti-virus and rhabdovirus have been studied for use in a gene therapy. However, these viral vectors have some problems. For instance, the most commonly used adenovirus has problems in that its manipulation is difficult and it causes an immune response in the human body. Accordingly, there has been a need for the development of a novel gene carrier for replacing the existing viral vectors.

Baculovirus is an insect cell-specific virus, and has been researched as a new viral vector system for a gene therapy owing to its advantage of not proliferating in a mammalian cell (see Boyce, F. M. and N. L. Bucher, Proc. Natl. Acad. Sci., USA 93: 2348-2352 (1996); Gronowski, A. M., et al., J. Virol., 73: 9944-9951 (1999); and Shoji I., H. et al., J. Gen. Virol., 78: 2657-2664 (1997)), with increasing applications. In addition, it has been reported that a baculoviral vector showed superior infection efficiency to adenovirus and were also safer than retrovirus or herpes simplex virus when administered to the human body (see Sandig V. and M. Strauss, J. Mol. Med., 74: 205-212 (1996)). Specifically, a recombinant baculovirus having VSVG as a pseudotype envelope (Huser et al., Nat. Biotechnol., 19: 451-455 (2001); Kalajzic et al., Virology, 284: 3745 (2001); and Ory et al., Proc. Natl. Acad. Sci., USA 93: 11400-11406 (1996)) causes infection in a cell through the mechanism of endocytosis and has an advantage in that infection occurs in non-dividing cells as well as in dividing cells (see Muramatsu et al., Biochem. Biophys. Res. Commun., 233: 45-49 (1997); and Ogawa et al., Int. J. Dev. Biol., 41: 111-122 (1997)).

The present inventors have endeavored to develop a viral vector with excellent gene delivery efficiency, and have found that a recombinant baculoviral vector comprising a VSVG gene and a PTD gene, which is a part of HIV-1 Tat gene, shows very high gene delivery efficiency.

### SUMMARY OF THE INVENTION

Accordingly, it is a major object of the present invention to provide a novel baculoviral vector having excellent gene delivery efficiency.

In accordance with one aspect of the present invention, there is provided a recombinant baculoviral vector comprising a VSVG gene and a PTD gene which is a part of HIV-1 Tat gene.

### BRIEF DESCRIPTION OF THE DRAWINGS

The above and other objects and features of the present invention will become apparent from the following description of the invention, when taken in conjunction with the accompanying drawings, which respectively show:

FIG. 1: a schematic diagram showing the structure of the inventive recombinant baculoviral vector, pBacG-PTD-EGFP-CMV.

FIG. 2: a schematic diagram displaying the process for preparing the inventive recombinant baculoviral vector, pBacG-PTD-EGFP-CMV.

FIG. 3: a microscopic picture exhibiting the expression of VSVG in the insect cell line Sf9 infected with the inventive baculoviral vector, pBacG-PTD-EGFP-CMV.

### DETAILED DESCRIPTION OF THE INVENTION

The recombinant retroviral vector of the present invention can be prepared by inserting VSVG gene and PTD gene, preferably the PTD derived from HIV-1 Tat gene, into the commercially available baculoviral vector, e.g., pBlueBac4.5 (Invitrogen Inc., USA).

In addition, the retroviral vector of the present invention may further comprise a EGFP (enhanced green fluorescent protein) gene for the confirmation of gene expression, and a cytomegalovirus (CMV) promoter to control the expression of the above mentioned genes.

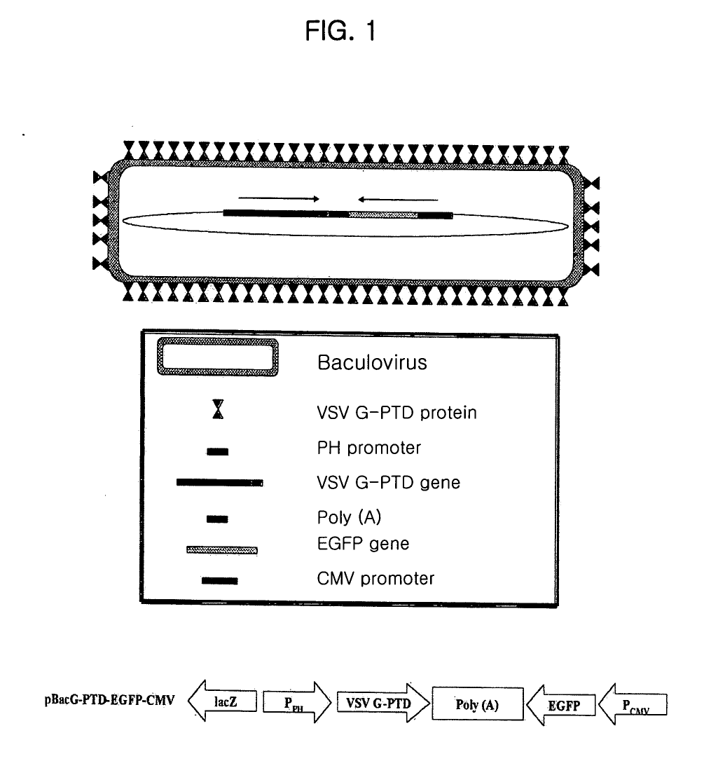

A preferred vector of the present invention is the recombinant baculoviral vector, pBacG-PTD-EGFP-CMV, which comprises a polyhedrin (PH) promoter of baculovirus, a VSVG gene, a PTD gene which is a part of HIV-1 Tat gene, a polyadenosine (poly(A)) sequence, a cytomegalovirus (CMV) promoter, and a EGFP gene, as shown in FIG. 1.

The inventive recombinant baculoviral vector is useful in a gene therapy due to its excellent efficiencies in the delivery of a target gene to the host cell and expression of the target gene in the host cell.

The following Examples are intended to further illustrate the present invention without limiting its scope.

### EXAMPLE 1

### Manufacture of Baculoviral Vector pBacG-PTD-EGFP-CMV

A polymerase chain reaction (PCR) (94° C., 5 minutes; 94° C., 1 minute, 57° C., 1 minute and 72° C., 1 minute (30 cycles); and 72° C., 5 minutes) was conducted with employing BacG-AFP-Luc+ (Park et al., Biochemcal Biophysical and Research Communications, November 30; 289(2):444-5 (2001)) as a template and a primer set of VSVGF (including HinIII recognition site) having the nucleotide sequence of SEQ ID NO: 1 and VSVGR (including Bg/II recognition site) having the nucleotide sequence of SEQ ID NO: 2 to amplify VSVG gene of 1,665-bp size. The amplified DNA was TA cloned into vector pCR2.1TOPO (Invitrogen Inc., USA) of 3.9 kb size to obtain vector pCR2.1TOPO-VSVG including VSVG gene.

Meanwhile, a double stranded DNA oligomer of 44-bp size, consisting of PTDF1 (including Bg/II recognition site) having the nucleotide sequence of SEQ ID NO: 3 and PTDR1 (including Sa/I recognition site) having the nucleotide sequence of SEQ ID NO: 4, was synthesized as a PTD gene. Then, pCR2.1TOPO-VSVG vector prepared above was cut with restriction enzymes HindIII and Bg/II to obtain VSVG gene therefrom, which were then ligated with the PTD gene to produce a VSVG-PTD fusion gene. A PCR (94° C., 5 minutes; 94° C., 1 minute, 57° C., 1 minute and 72° C., 1 minute (30 cycles); and 72° C., 5 minutes) was conducted with employing the VSVG-PTD fusion gene as a template and primers having the nucleotide sequences of SEQ ID NOs: 1 and 4. The VSVG-PTD fusion gene thus amplified was TA cloned into pCR2.1TOPO vector to obtain pCR2.1TOPO-VSVG-PTD vector. The pCR2.1TOPO-VSVG-PTD vector was cut with XhoI and Sa/I to produce a VSVG-PTD fragment, which was then inserted into XhoI/Sa/I cut site of pBlueBac4.5 vector (Invitrogen Inc., USA) to obtain pBacG-PTD vector.

Meanwhile, pEGFP-N vector (Clontech Inc., USA) of 4.7-kb size was cut with KpnI and NotI restriction enzymes to produce a EGFP gene fragment of 0.8-kb size, which was then inserted into KpnI/NotI cut site of pCEP4 vector (Invitrogen Inc., USA) of 10.4-kb size to produce pCEP4-EGFP vector. The pCEP4-EGFP vector was cut with Sa/I to separate a CMV-EGFP gene fragment therefrom. The CMV-EGFP gene fragment was subjected to blunt-ended ligation at SnaBI cut site of pBacG-PTD vector to obtain pBacG-PTD-EGFP-CMV vector. Fill-in reaction was conducted by employing T4 DNA polymerase (New England Biolabs Inc., Beverly, Mass.) in accordance with the manufacturer's instruction.

The structures of the prepared plasmid vectors were confirmed by cutting them with appropriate restriction enzymes (New England Biolabs Inc.), followed by an electrophoresis on 0.7% agarose gel.

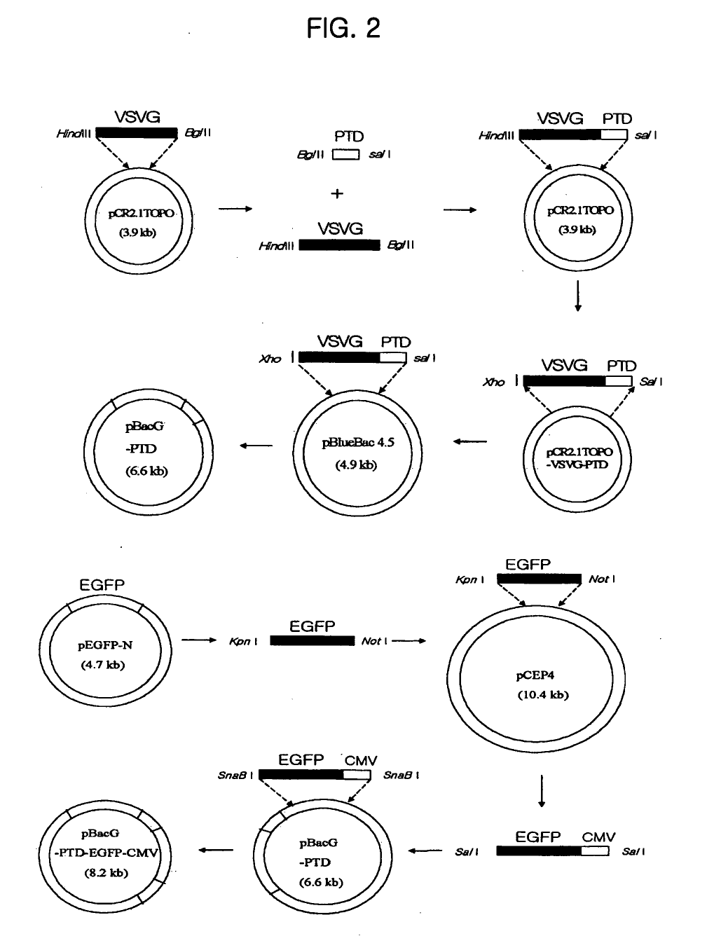

FIG. 1 shows a schematic diagram of the structure of baculoviral vector pBacG-PTD-EGFP-CMV, and FIG. 2, a schematic diagram of the manufacturing process thereof.

### EXAMPLE 2

### Examination of the Expression of VSVG in the Insect Cell Line Sf9

Insect cell line Sf9 (Invitrogen Inc., USA) cells were infected with baculoviral vector pBacG-PTD-EGFP-CMV prepared in Example 1. The Sf9 cells were then cultured for three days in Graces insect medium (GIBCO Inc., USA) containing 10% fetal bovine serum at 37° C. under 5% CO₂-95% air. Degree of the expression of VSVG protein was observed by a fluorescent microscope.

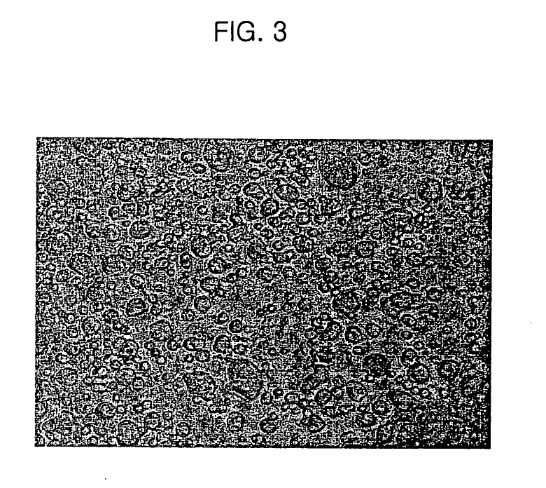

As shown in FIG. 3, the cells not infected with the inventive recombinant baculoviral vector, pBacG-PTD-EGFP-CMV, were observed as small spheres, while the cells infected with the inventive baculoviral vector were observed in the form of bigger amorphous cells. When VSVG protein is expressed in a recombinant virus as a pseudotype envelope, it moves to the cell membrane and induces cell fusion, thereby increasing the size of cell. This phenomenon was observed in the present example by employing the cells infected with the baculoviral vector of the present invention.

While the invention has been described with respect to the above specific embodiments, it should be recognized that various modifications and changes may be made to the invention by those skilled in the art which also fall within the scope of the invention as defined by the appended claims.

1. A recombinant baculoviral vector comprising a vesicular stomatitis virus glycoprotein (VSVG) gene and a protein transduction domain (PTD) gene derived from Tat gene of human immunodeficiency virus-1 (HIV-1).

2. The recombinant baculoviral vector of claim 1, which comprises a polyhedrin (PH) promoter of baculovirus, a VSVG gene, a PTD gene derived from HIV-1 Tat gene, a polyadenosine (poly(A)) sequence, a cytomegalovirus (CMV) promoter, and an EGFP (enhanced green fluorescent protein) gene in sequence.

3. The recombinant baculoviral vector of claim 1, wherein the vector is pBacG-PTD-EGFP-CMV.


SEQUENCE LISTING (4 sequences)

SEQ ID NO: 1 - DNA, 32 bp, Artificial Sequence
  VSVGF primer
agctaagatctctttccaagtcggttcatctc

SEQ ID NO: 2 - DNA, 32 bp, Artificial Sequence
  VSVGR primer
agatcaaagcttcactatgaagtgccttttgt

SEQ ID NO: 3 - DNA, 44 bp, Artificial Sequence
  oligomer petide PTDF1 DNA
gatcttatggcaggaagaagcggagacagcgacgaagataactg

SEQ ID NO: 4 - DNA, 44 bp, Artificial Sequence
  oligomer petide PTDR1 DNA
tcgacagttatcttcgtcgctgtctccgcttcttcctgccataa